(19) United States
(12) Patent Application Publication
(10) Pub. No.: US 2023/0337903 A1
(43) Pub. Date: Oct. 26, 2023

(54) COAXIAL DILATION SPECULUM APPARATUS AND METHOD TO ACCESS, ILLUMINATE, AND VISUALIZE.

(71) Applicants: Yvonne Wolny, Chicago, IL (US); Martinho Pais, North Barrington, IL (US)

(72) Inventors: Yvonne Wolny, Chicago, IL (US); Martinho Pais, North Barrington, IL (US)

(21) Appl. No.: 18/297,479

(22) Filed: Apr. 7, 2023

Related U.S. Application Data
(60) Provisional application No. 63/328,860, filed on Apr. 8, 2022.

(51) Int. Cl. A61B 1/32; A61B 1/303; A61M 3/02

(52) U.S. Cl. CPC A61B 1/32; A61B 1/303; A61M 3/0291; A61M 2210/1475

(57) ABSTRACT

Device and method are disclosed for use in a medical-clinic office and surgical settings for examination and treatment. A device to access human vaginal canal in the form of a coaxial quasi-cylindrical dilation apparatus with features that enable adjustment of diameter and depth of access into the anatomical area and a light source to visualize the entire area of interest. A method to access and visualize a human canal using a cylindrical expandable apparatus with an attached light source for enhanced visualization of the entire area. To irrigate and clear the surfaces, a method to introduce and evacuate a jet of gas or spray of liquid, is disclosed.

### BACKGROUND

### Field of the Invention

The disclosed invention relates to a device and method to internally dilate, traverse, illuminate, and visualize a human canal. More particularly, the device relates to accessing the vaginal canal in the form of a coaxial cylindrical dilation device with features that facilitate the adjustment of the diameter and the depth of penetration into the area of interest. A method to illuminate is integrated into the device, so as to visualize both the internal side walls of the vaginal canal and the cervix. Furthermore, a method to introduce and evacuate using a jet of gas or spray of liquid to irrigate and clear the surfaces of the vaginal canal of any refuse or secretions, is disclosed.

### Background Art

Vaginal speculums are widely utilized in obstetrics and gynecology for the purpose of examination and treatment. To be able to perform an adequate and unobstructed examination of the vaginal canal it is paramount to have unobstructed visual access to the exterior and entire internal region of the vagina, constituting the lateral vaginal walls and the front face of the cervix. Irrigation and removal of excess secretions in the anatomical area encompasses an important part of a successful examination and treatment.

The vaginal canal is partly cylindrical and partly curved in the lengthwise direction, see FIG. 1 and FIG. 2. The proximal entrance to the vaginal canal is called the vaginal introitus. At its distal end, the vaginal canal ends internally at the cervix that constitutes the proximal part of the uterus protruding into the distal end of the vaginal canal. Thus, any instrument that is used to enter and dilate the vaginal canal must have the capability to navigate through and accommodate to its inherent irregular geometry. Currently used speculums, example see FIG. 3, comprise two blades, an upper blade and a lower blade with a handle, which when using a lever mechanism, the “thumb hinge”, allows for expanding the area of the vaginal canal in the upper and lower directions. The thumb screw facilitates holding the set position. However, in some patients, depending on their anatomy, the lateral walls of the vaginal canal are not expanded sufficiently, inducing the sidewalls to cave in, thus obstructing the view. In the event, where the patient needs gynecological procedures to be performed utilizing electrocautery or laser, the need for an unobstructed view is essential, demanding some means to prevent collapse of the lateral vaginal walls to enable proper visualization and limit a risk from injury due to unintended exposure of the tissue to the surgical procedures and types of energy used, viz. electrocautery, laser. Further, this is also particularly important in obstetrical diagnostic-operative procedures dealing with access to the fetal and maternal tissues.

In certain circumstances associated with anatomy of the patient, there is a need to be able to access more distant interior and angled areas of the vaginal canal by the operator-physician. An additional limitation of present commercially used speculums is the length of the blades, which cannot be extended beyond the preset manufactured fixed length. Based on the above, there is a need for a speculum that supports a variable diameter and depth of insertion. Depending on the anatomy of the vaginal canal and the position of the cervix, a more flexible speculum would be beneficial for a thorough examination and treatment. The combination of an adjustable length and diameter of the speculum with lengthwise flexibility in curvature would be ideal for multiple applications in obstetrical and gynecological examinations-procedures where an unobstructed access to the cervix is especially crucial for locating and placement of surgical instruments. An unobstructed access to the cervix is especially paramount during the more advanced gynecological robotic-laparoscopic procedures enabling adequate placement of uterine manipulators by the operator.

The use of illumination is also available in certain current speculums, by configuring the speculum as a light-guide, see FIG. 4. The current design of the speculum has an upper blade and a lower blade with an attached global illumination system. Even though such a system provides general global illumination to the lateral vaginal walls and the cervix, it has no means to selectively illuminate or brighten the area of interest. In addition, the current speculum structures do not enable optimal visualization of the examination-surgical field because the lateral vaginal walls tend to collapse into the lateral internal open space between the blades when the speculum is expanded.

To limit the vaginal side wall collapse, attempts were made to utilize plastic or elastomeric cover sheets over the entire speculum with an opening at the proximal end. Placement of a cover sheet includes an additional step over uneven speculum surfaces prior to the exam with a possibility of slippage of the cover sheet during insertion and navigation. The cover sheet also prevents visualization of the lateral vaginal canal walls, which is an important aspect of the procedure. Even if there is a stretchable tubular coverage of the speculum, it may not allow for conformable expansion of the vaginal walls. Furthermore, the speculum does not include an illumination feature to provide better visualization of the area.

There is a need to have vaginal walls and cervix that are clean-clear of secretions-refuse and also have an unobstructed field of view while the speculum is used during examination-treatment. Hence, a feature providing irrigation and evacuation of secretions-refuse in the region of interest would be beneficial. Such a feature is not present in currently available speculums.

Therefore, there is a need for a vaginal speculum which enables the operator-physician to create an unobstructed view of the entire vaginal canal including the lateral vaginal walls and the cervix at the distal end. In addition, features facilitating illumination of the entire vaginal canal are needed to enable a safe examination and-or surgery. Finally, a means to clear and irrigate the internal vaginal surfaces of secretions-refuse is desired.

### SUMMARY OF THE INVENTION

The new and improved apparatus facilitates the dilation of an orifice-canal in the body of a human to allow for a clear illuminated visual inspection and unimpeded physical access to the orifice-canal with instruments for visualization, treatment, and surgery.

Specifically, the current invention is directed to a coaxial quasi-cylindrical dilation apparatus enabling expansion of the vaginal canal while conforming to the anatomy of the subject-patient. The cross-section of the vaginal canal is of irregular quasi-cylindrical shape while in length it may follow an irregular curve shape. Hence, this demands an apparatus that facilitates the dilation of the vaginal canal while automatically conforming to the allowable expandable shape of the tissue with minimum physical discomfort to the patient.

Radial expansion and conformance of the speculum is effectuated by a coaxial quasi-cylindrical squirrel cage structure with spring-like conforming resilience. The cage takes the form of a set of curvilinear beam(s) held together by quasi-cylindrical-spring(s). The above is configured so as to be in a contracted-retracted state to attain a diametrically smaller and compact speculum. Further, on insertion and release in the vaginal canal, the speculum expands radially to dilate and conform to the internal geometry of the vaginal canal.

The coaxial quasi-cylindrical shape of the cage apparatus prevents collapsing of the lateral walls of the vaginal canal into the examination-operative field enabling the operator-physician an unobstructed view of the vaginal canal region of interest. An additional benefit of the adjustable shape of the coaxial quasi-cylindrical apparatus includes the extended length of the apparatus beyond the length of current speculums available for use. The feature is especially beneficial for patients with longer vaginal canals where access using current speculums is limited. A further benefit of the coaxial quasi-cylindrical apparatus includes the capability of rotational movements to access asymmetric location of the target organ (cervix) in certain patients.

Adjustments of the diameter and the length of the coaxial quasi-cylindrical apparatus combined with illumination of the internal vaginal canal walls and the cervix provides the operator-physician with much better access to the area of interest in multiple clinical settings. In order to optimize the desired diameter of the apparatus, a quasi-cylindrical spring mechanism is implemented. In addition to achieving a desired diameter, the depth of insertion can be adjusted by sliding the cage apparatus in or out.

In addition to being useful in office settings, the apparatus can be used in surgical settings where adequate visualization of the surgical field is especially crucial while performing minimally invasive and advanced gynecological procedures.

In the field of obstetrics, an expandable coaxial quasi-cylindrical apparatus is also very useful taking into account the much looser tissue of the vaginal canal during pregnancy. To adequately visualize the cervix and the lateral vaginal walls, the coaxial quasi-cylindrical apparatus enables a clear view which combined with the attached light source provides excellent illumination of the lateral vaginal walls and the cervix.

To facilitate a smooth insertion of the coaxial quasi-cylindrical apparatus into the vaginal canal, a sheath-cover is placed on the contracted-retracted state of the coaxial quasi-cylindrical apparatus. Upon adequate positioning of the coaxial quasi-cylindrical apparatus with the sheath-cover in the vaginal canal, the sheath-cover is removed at its proximal end by pulling out the cover. The removal process is facilitated by inclusion of scribes at the distal end of the sheath-cover. Once the sheath-cover is removed, the contracted-retracted coaxial quasi-cylindrical apparatus expands in conformity to the desired length, diameter, and shape of the vaginal canal.

In addition to an unobstructed view of the area of interest, visualization of the vaginal canal and the cervix are facilitated by dual, independent high resolution digital cameras with corresponding adjustable intensity light sources. For that purpose, one camera is facing the front-axial direction providing an axial view specifically that of the cervix. The second camera is radial-side facing, providing a view of the lateral vaginal walls.

The system supports viewing the captured images of the front and lateral views via connecting over Wi-Fi, Bluetooth, wired USB, etc. to operator-physician selectable display devices including a mobile device, computer, smart TV, or AR-VR glasses. This feature is especially useful in obstetrical and surgical settings. Optical and-or digital magnification of the area of interest is also supported. Finally, the system includes the option to inject gas and-or liquids for the purpose of clearing the area being examined or operated on of refuse-secretions.

The method facilitates the operator-physician with the ability to dilate the region of interest, traverse, and view the vaginal wall and the cervix while introducing instrumentation to perform necessary diagnostic and therapeutic procedures. Adjustments of the diameter and the depth of insertion into the vaginal canal, enables procedures in challenging clinical scenarios where the region of interest is out of the range of currently available speculums. Further, the method enhances visualization of the area of interest providing illumination to the front and lateral walls of the vaginal canal followed by capture of the images on dual high-resolution cameras.

### BRIEF DESCRIPTION OF THE DRAWINGS

FIG. 1 is a frontal view of the pelvic region of a female.

FIG. 2 is a side view of the pelvic region of a female.

FIG. 3 is a perspective view of a speculum with a handle and two blades.

FIG. 4 is a frontal view of a two-blade plastic speculum with an attached light source.

FIG. 5 is a geometrical representation of the coaxial quasi-cylindrical dilation apparatus and mechanism.

FIG. 6 is a perspective and axial view of the coaxial quasi-cylindrical corrugated monocoque spring with a curvilinear beam in the expanded state.

FIG. 7 is a perspective and axial view of the coaxial quasi-cylindrical corrugated monocoque spring with a curvilinear beam in a contracted-retracted state.

FIG. 8 is a perspective and axial view of a sheath-cover with hemispherical tip at the distal end with scribes.

FIG. 9 is a perspective view of a sheath-cover where the hemispherical tip is ruptured at the scribes.

FIG. 10 is a perspective and axial view of the coaxial quasi-cylindrical corrugated monocoque spring with a curvilinear beam in a retracted state held inserted in a sheath-cover.

FIG. 11 is a perspective side view of the coaxial quasi-cylindrical dilation apparatus with curvilinear beams external to the quasi-cylindrical springs in an expanded state.

FIG. 12 is a perspective and axial view of the coaxial quasi-cylindrical dilation apparatus with curvilinear beams external to the quasi-cylindrical springs in a contracted-retracted state.

FIG. 13 is a perspective and axial view of the coaxial quasi-cylindrical dilation apparatus with curvilinear beams external to the quasi-cylindrical springs in a contracted-retracted state inserted in a sheath-cover.

FIG. 14 is a perspective and side view of the coaxial quasi-cylindrical dilation apparatus with curvilinear beams internal to the quasi-cylindrical springs in an expanded state.

FIG. 15 is a perspective and axial view of the coaxial quasi-cylindrical dilation apparatus with curvilinear beams internal to the quasi-cylindrical springs in a contracted-retracted state.

FIG. 16 is a perspective and axial view of the coaxial quasi-cylindrical dilation apparatus with curvilinear beams internal to the quasi-cylindrical springs in a contracted-retracted state inserted in a sheath-cover.

FIG. 17 is a perspective and axial view of a camera module.

FIG. 18 is a perspective view of a camera module-instrument support clip.

FIG. 19 is a perspective and axial view of a camera module with support clips.

FIG. 20 is a perspective and axial view of a camera module attached to the coaxial quasi-cylindrical corrugated monocoque spring with a curvilinear beam in the expanded state.

FIG. 21 is a perspective and axial view of a camera module attached to the coaxial quasi-cylindrical dilation apparatus with curvilinear beams external to the quasi-cylindrical springs in an expanded state.

FIG. 22 is a perspective and axial view of a camera module attached to the coaxial quasi-cylindrical dilation apparatus with curvilinear beams internal to the quasi-cylindrical springs in an expanded state.

FIG. 23 is a relation between the size and shape of the curvilinear beam and the stress induced in its material makeup.

FIG. 24 is a relation between the size and shape of the curvilinear beam and the level of bend-deflection induced in it due to the forces imposed by the lateral vaginal walls.

FIG. 25 is a flowchart of method of assembly and insertion of the coaxial quasi-cylindrical dilation apparatus.

FIG. 26 is a flowchart of method of insertion and removal of the camera module.

FIG. 27 is a flowchart of method of removal of the coaxial quasi-cylindrical dilation apparatus.

### DETAILED DESCRIPTION OF THE DRAWINGS

The present disclosure, herein referred to “Co-axial Dilation Speculum Apparatus”, is to be considered as an exemplification of the invention and is not intended to limit the invention by illustrations of the embodiments or their description. It should be noted that there are many variations and modifications of the apparatus and method disclosed here that would be apparent to one skilled in the art, and that are within the scope of the inventive concepts.

Embodiments of the described invention provide various novel apparatus and improvements over available speculums allowing adjustable expansion of the entire vaginal canal, visualization of the lateral walls of the vagina canal, and the cervix. Illumination of the entire area is permitted by attachment of an integrated camera and a light source apparatus to the speculum apparatus. In addition, the camera enables magnification of the region of interest. Elimination of refuse-secretions present in the vaginal canal is effectuated by a feature supporting injection of a jet-spray of liquid-water or jet of gas and subsequent evacuation of the refuse in the vaginal canal.

The coaxial dilation speculum apparatus with a sheath-cover, facilitates means to navigate, translate, and rotate the speculum within the vaginal canal, means to actuate the level of dilation in the radial direction in conformity with the geometry of the vaginal canal, means of imaging with zoom and focus capability, means to translate the imaging module down the length of the canal in the axial direction so as to view straight down, means to rotate the imaging device to view the side walls of the vaginal canal at any axial location, light sources that illuminates in the axial and sideways directions with brightness control, and imaging module that remotes the image via Wi-Fi, BT, wired-USB to an operator-physician selectable display device. The remote display device includes combination of a mobile device, computer, smart TV, or AR-VR glasses. Also, integrated into the imaging apparatus is a feature to support injecting a jet-puff of gas to clear the surfaces being viewed, and a feature to support injecting a jet-spray of liquid to clear the surfaces being viewed.

The coaxial dilation speculum apparatus has structural features that enables rotation in the angular direction, contraction, and dilation in the radial direction. Further it is long enough so as to be inserted into the vaginal canal with the distal end to be juxtaposed with the cervix and the proximal end extending external to the introitus sufficiently so as to provide a means to hold by the operator-physician. The navigation of the speculum is envisaged to be a simple, single-hand driven to enable rotation and translation in a back-forth action.

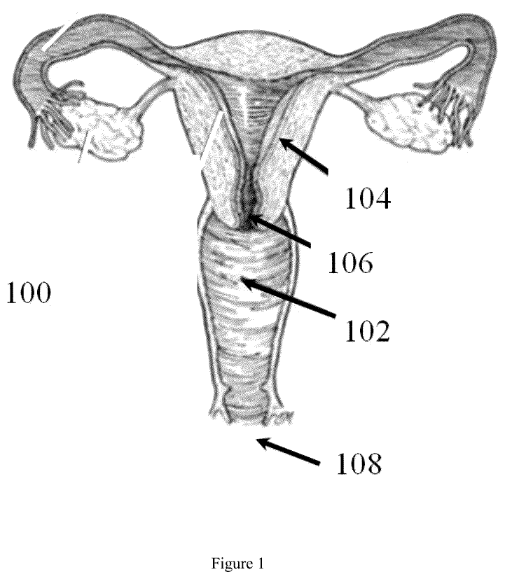

Referring to FIG. 1, it illustrates a front view of the human female pelvis 100 showing representative anatomy that includes the vaginal canal 102, the uterine corpus 104, and the cervix 106. The anatomy of the vaginal canal 102 entails a longitudinal canal extending from the opening-introitus 108 to the cervix 106. The vaginal canal 102 has a tubular shape with varying, non-uniform diameter throughout the entire length. The vaginal canal 102 is primarily composed of muscle and connective tissue. Its size, i.e., length, diameter, and thickness of wall, are highly dependent on the age and anatomy of the patient. Generally, the introitus 108 region has a narrower diameter than the midsection and the distal and proximal end of the vaginal canal. The female cervix 106 constitutes the proximal part of the uterine corpus 104 that protrudes into the distal end of the vaginal canal 102. As illustrated in FIG. 1, the diameter of the cervix 106 is narrower than the distal end of the vaginal canal 102 which can vary depending on the age, parity, uterine pathology (uterine fibroids or endometriosis), anatomical variations (anteverted or retroverted uterus), or prior surgical procedures. Upon insertion of the speculum, the diameter of the introitus is expanded to facilitate insertion of the apparatus. In addition to visualization of the lateral walls of the vaginal canal 102, the main purpose of utilizing a vaginal speculum is to visualize and have physical instrument access to the much narrower in diameter, cervix 106. As such, the speculum trajectory and shape needs to conform to the conical shape that is wider at the proximal end of the vaginal canal (introitus) than at the distal end juxtaposed to the cervix 106.

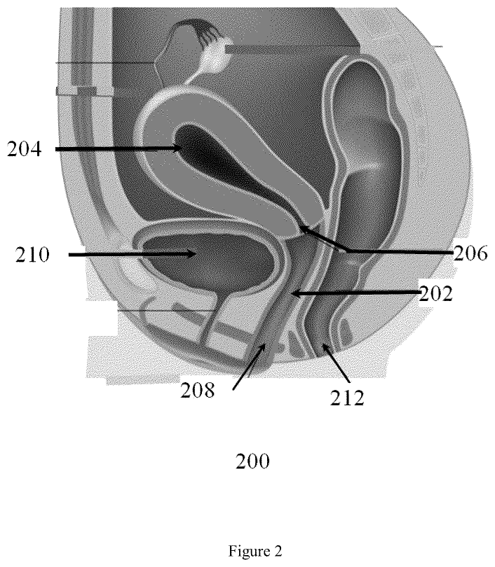

FIG. 2 represents a lateral view of the human female pelvis 200 that includes the vaginal canal 202, the uterine corpus 204, the cervix 206, the introitus 208, the bladder 210, and the rectum 212. The vaginal canal 202 is primarily composed of muscle and connective tissue. Its size, i.e., length, diameter, and thickness of wall, are highly dependent on the age and anatomy of the patient. Generally, the introitus 208 region has a narrower diameter than the midsection, the distal and proximal end of the vaginal canal 202. The female cervix 206 constitutes the proximal part of the uterine corpus 204 that protrudes into the distal end of the vaginal canal 202. The varied length and diameter of the vaginal canal 202 demands a speculum that is not only variable in diameter and in length, but also flexible in shape to conform to the shape of the vaginal canal 202. The primary purpose of use of the speculum is not only visual access to the vaginal walls 202 and the cervix 206, but also an unobstructed instrument access for examination and surgical procedures. Hence, there is also a need for a speculum to realize and support a desirable level of dilation of the vaginal canal 202.

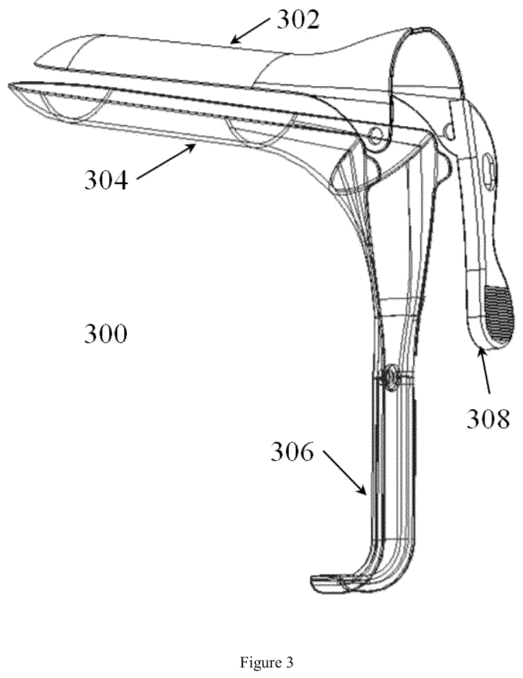

FIG. 3 represents an example “duckbill” configuration of a currently used speculum 300. Speculum 300 comprises two blades, an upper blade 302 and a lower blade 304 with a handle 306, which when using a lever mechanism in the form of a thumb operated hinge 308, allows for expanding the area of the vaginal canal in the upper and lower directions. Even though the vaginal canal is dilated in the anterior and posterior directions and physically supported, depending on the anatomy of the patient, there is a possibility of the lateral-side walls of the vaginal canal 102 caving-folding inwards and obstructing the view.

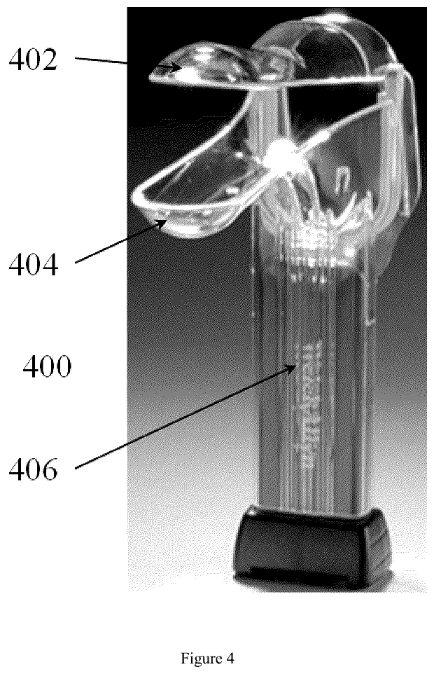

The use of illumination is also available in certain current speculums, by configuring the speculum as a light-guide, see FIG. 4. An existing design of a speculum 400 has an upper blade 402 and a lower blade 404 with an attached illumination system 406. Even though such a system provides general illumination to the lateral vaginal walls and the cervix it has no means to selectively illuminate or brighten the area of interest. Further, the current speculum 400 does not provide means to eliminate any refuse-secretions that accumulate, especially at the distal end of the area of interest (cervix 106). Further, the location of the illumination system at the proximal end of the existing speculum 400 creates a glare from the light that is reflected proximally towards the operator-physician, and thus decreases the intensity of the illumination at the desired target.

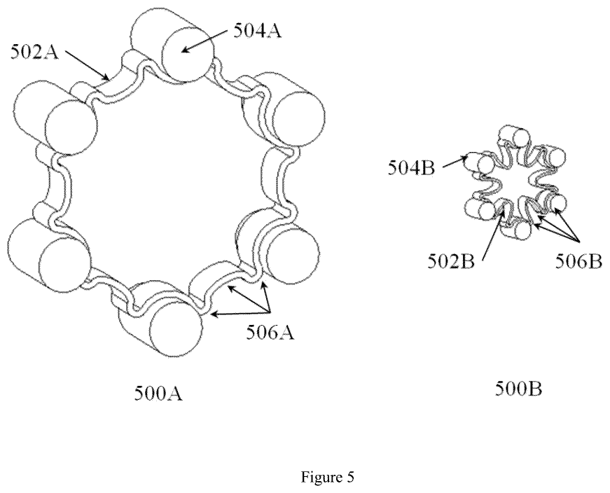

FIG. 5 illustrates the general principle of operation of the underlying mechanism of the “coaxial quasi-cylindrical dilation apparatus”. A class of materials exist that inherently have elastic properties, implying that if objects made from them are stretched or compressed within certain limits, they will return to their original dimensions and shape when the forces are removed; in essence they can be considered a spring. The design of the coaxial quasi-cylindrical spring is equivalent to a circular spring with certain features added to the design to facilitate supporting the load. 500A illustrates a geometrically featured coaxial quasi-cylindrical spring in the expanded state. The coaxial quasi-cylindrical spring 502A has features that allow for attachment of curvilinear beam elements 504A that serve the purpose to transfer the radially outward spring-force longitudinally to the external load. In this application the external load are the walls of the vaginal canal 102. 500B illustrates the geometrically featured coaxial quasi-cylindrical spring of 500A in the contracted-retracted state. Further, the coaxial quasi-cylindrical spring design has geometrical features 506A-506B built in that induces it to buckle and fold inwards into a compact form when a radially inward compressive force is applied to the curvilinear beams 504B. When released, the contracted coaxial quasi-cylindrical spring 502B expand to push the curvilinear beams 504B radially outward against the lateral walls of the vaginal canal 102, thus dilating the vaginal canal. In this embodiment, the curvilinear beams 504A are captured external to the coaxial quasi-cylindrical spring 502A as illustrated in FIG. 5. Here the coaxial quasi-cylindrical spring 502A and curvilinear beam 504A geometries and numbers are selected to spread the dilation load so as to reduce pressure concentration on the vaginal canal 102 tissues. Circular curvilinear beam 504A geometries, six in number are presented to illustrate feasibility. It is understood that other curvilinear beam geometries and numbers may also be utilized without departing from the spirit or scope of the present disclosure. The description is, therefore, not to be taken in a limiting sense.

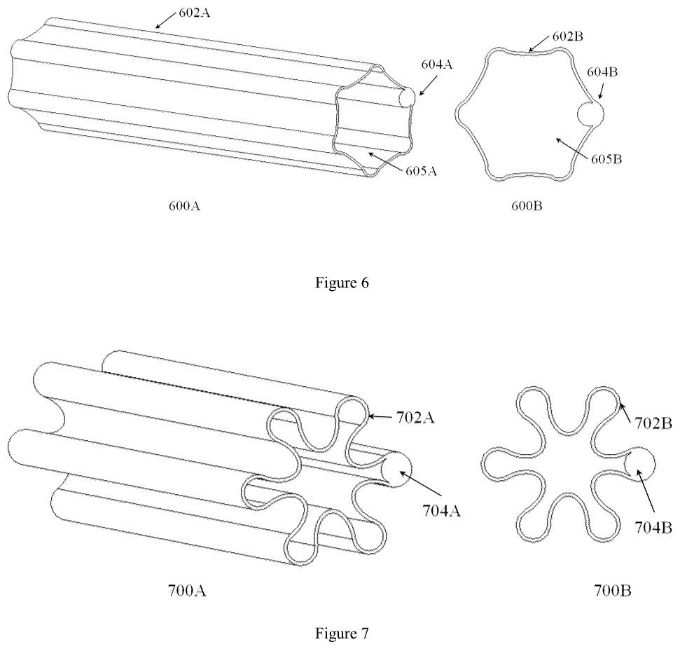

Referring to FIG. 6, is illustrated the coaxial dilation speculum apparatus 600 in a perspective view 600A and an axial view 600B with a quasi-cylindrical corrugated monocoque spring 602A-602B to which is attached a curvilinear beam 604A-604B that extends the entire length of the quasi-cylindrical corrugated monocoque spring 602A-602B. In this embodiment, the curvilinear beam 604A-604B is captured internal to the quasi-cylindrical corrugated monocoque spring 602A-602B. The assembly 600 is illustrated in the expanded configuration. In this embodiment, the monocoque spring 602-602B takes the form of a conformable cylindrical cage that facilitates a uniform circumferential and longitudinal dilation of the lateral vaginal walls 102. The monocoque springs 602A-602B on expanding radially creates a radial force to support the collapsing forces of the vaginal wall 102 into the field of vision of the speculum internal void 605A-605B while enabling an unobstructed view and path for the operator-physician managed instruments to probe and operate on the vaginal canal 102 and the cervix 106.

The vaginal canal 102, is a muscular quasi-cylindrical tissue which is highly elastic and in a contracted state under normal conditions. To introduce the speculum into the introitus 108 the speculum 600 needs to be also apriori contracted-retracted. Referring to FIG. 7, it illustrates the coaxial dilation speculum apparatus 600 in a contracted-retracted state 700 in a perspective view 700A and an axial view 700B with a quasi-cylindrical corrugated monocoque spring 702A-702B to which is attached a curvilinear beam 704A-704B that extends the entire length of the quasi-cylindrical corrugated monocoque spring 702A-702B. The curvilinear beam 704A-704B serves the purpose of facilitating capture and guidance of instruments to be described later in the description.

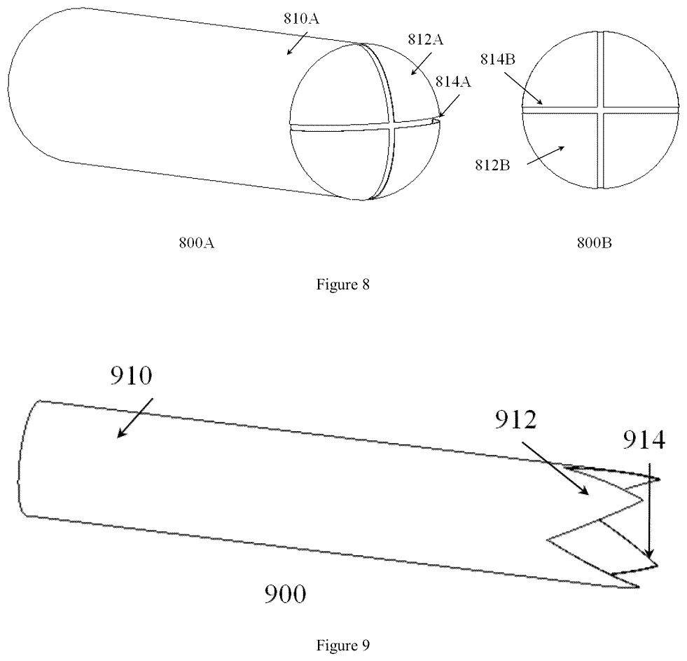

The coaxial dilation speculum apparatus 700 is held in the contracted-retracted state by inserting it into a cylindrical sheath-cover with a hemispherical scribed tip 800 as illustrated in FIG. 8, in a perspective view 800A and an axial view 800B. The sheath-cover constitutes a thin-walled cylindrical tube 810A, open at the proximal end and has a partially closed spherical tip 812A-812B at the distal end. The spherical tip 810A-810B enables smooth insertion into the introitus 108 and navigation in the vaginal canal 102. To further facilitate smoother insertion and minimal discomfort to the patient, a lubricant is applied to the external surface of the sheath-cover 800. The spherical tip 812A-812B is further scribed 814A-814B. The scribe features 814A-814B induce a local structural weakness in the spherical region of the material that on the application of an appropriate axial pull from the proximal direction will facilitate initiation of a tear-rupture of the hemispherical tip 812A-812B at predetermined junctions 814A-814B to facilitate ease of removal of the sheath-cover in the proximal direction.

FIG. 9, in a perspective view 900 of the cylindrical sheath-cover 910 with the ruptured hemispherical tips 912 at the scribes 914 on removal. The cylindrical sheath cover with a hemispherical tip 800A-800B is a one-time use and disposable on removal 900. A spherical tip 812A-812B is suggested here to facilitate smooth insertion into the introitus 108. Similarly, the spherical tip 812A-812B has suggested four scribes to initiate rupture and ease of removal. The spherical geometry and four number of scribes are presented to illustrate feasibility. It is understood that other tip geometries and scribe numbers may also be utilized without departing from the spirit or scope of the present disclosure. The description is, therefore, not to be taken in a limiting sense.

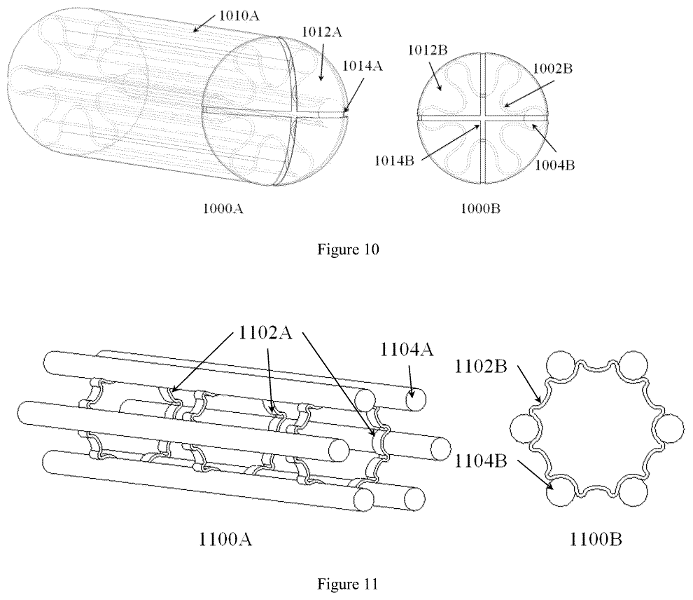

FIG. 10 illustrates an assembly of the coaxial dilation speculum apparatus 700A-700B inserted into the sheath-cover 800A-800B in a perspective view 1000A and an axial view 1000B that comprises quasi-cylindrical-monocoque spring 1002B with an internally captured curvilinear beam 1004B in a contracted-retracted state inserted in a cylindrical sheath-cover 1010A with a hemispherical tip 1012A-1012B that is locally scribed 1014A-1014B. After the operator locates, adjusts, and places the speculum assembly 1000A-1000B into the vaginal canal 102, the cylindrical sheath-cover with a hemispherical tip 1010A-1012A is removed. Upon removal of the sheath cover, the coaxial dilation speculum apparatus expands due to the release of the compressed spring force and dilates the vaginal canal 102 in conformity with its anatomy, thus enabling an unobstructed view and path for operator-physician managed instruments to probe and operate on the vaginal canal 102. and the cervix 106. Here the quasi-cylindrical-monocoque spring 1002B and beam 1004B geometries are selected to spread the dilation load so as to reduce pressure concentration on the vaginal canal 102 tissues. Cylindrical curvilinear beam 1004B geometries, one in number are presented to illustrate feasibility. It is understood that other curvilinear beam geometries and numbers may also be utilized without departing from the spirit or scope of the present disclosure. Similarly, a quasi-cylindrical-monocoque spring 1002B with six lobe geometry is presented to illustrate feasibility. It is understood that other monocoque geometries and lobe numbers may also be utilized without departing from the spirit or scope of the present disclosure. The description is, therefore, not to be taken in a limiting sense.

In another embodiment, FIG. 11, illustrates the coaxial dilation speculum apparatus in a perspective view 1100A and an axial view 1100B with three quasi-cylindrical springs 1102A-1102B coupled to externally captured curvilinear beams 1104A-1104B. The coaxial dilation speculum apparatus 1100A-1100B is illustrated in the expanded configuration. The coaxial dilation speculum apparatus 1100A-1100B being a conformable cylindrical cage constructed of three quasi-cylindrical springs 1102A-1102B that radially support six longitudinally running curvilinear beams 1104A-1104B. This assembly in effect dilates and supports, radially and longitudinally, the surrounding vaginal walls 102 while enabling an unobstructed view and path for operator-physician managed instruments to probe and operate on the vaginal canal 102 and the cervix 106.

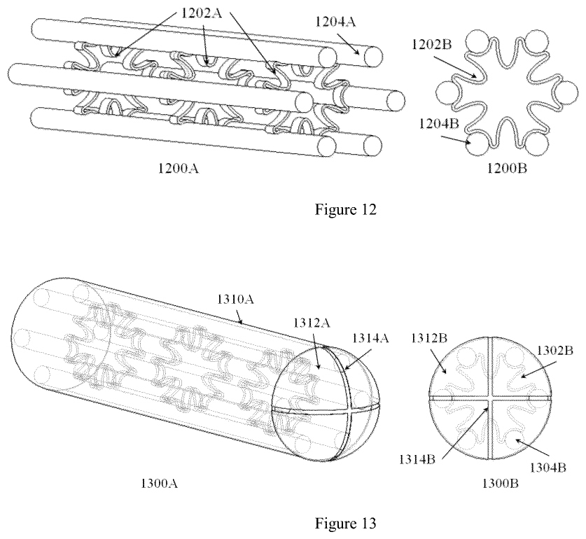

The vaginal canal 102, is a muscular quasi-cylindrical tissue which is highly elastic and in a contracted state under normal conditions. To introduce the speculum into the introitus 108 the speculum 1100A-1100B needs to be also apriori contracted-retracted. Referring to FIG. 12, it illustrates the coaxial dilation speculum apparatus 1100A-1100B in a contracted-retracted state in a perspective view 1200A and an axial view 1200B with three quasi-cylindrical springs 1102A-1102B coupled to six externally captured curvilinear beams 1104A-1104B. Over and above supporting the walls of the vaginal canal 102, the curvilinear beams 1104A-1104B also serve the additional purpose of facilitating capture and guidance of instruments to be described later in the description.

The coaxial dilation speculum apparatus 1100A-1100B is held in the contracted-retracted state by inserting it into a cylindrical sheath-cover with a hemispherical scribed tip 800 as illustrated in FIG. 8, in a perspective view 800A and an axial view 800B. FIG. 13 illustrates an assembly of the coaxial dilation speculum apparatus 1200A-1200B inserted into the sheath-cover 800A-800B in a perspective view 1300A and an axial view 1300B that comprises three quasi-cylindrical springs 1302B coupled to six externally captured curvilinear beams 1304B in a contracted-retracted state inserted in a cylindrical sheath-cover 1310A with a hemispherical tip 1312A-1312B that is locally scribed 1314A-1314B. After the operator locates, adjusts, and places the speculum apparatus assembly 1300A-1300B into the vaginal canal 102, the cylindrical sheath cover with a hemispherical tip 1310A-1312A is removed. Upon removal of the sheath cover, the coaxial dilation speculum expands due to the release of the contracted-retracted spring force and dilates the vaginal canal 102 in conformity with its anatomy, thus enabling an unobstructed view and path for operator-physician managed instruments to probe and operate on the vaginal canal 102 and the cervix 106. Here the quasi-cylindrical springs 1302B and curvilinear beams 1304B geometries are selected to spread the dilation load so as to reduce pressure concentration on the vaginal canal 102 tissues. Circular curvilinear beam 1304B geometries, six in number are presented to illustrate feasibility. It is understood that other beam geometries and numbers may also be utilized without departing from the spirit or scope of the present disclosure. Similarly, the three quasi-cylindrical springs 1302B with six lobe geometry is presented to illustrate feasibility. It is understood that other quasi-cylindrical spring geometries and lobe numbers may also be utilized without departing from the spirit or scope of the present disclosure. The description is, therefore, not to be taken in a limiting sense.

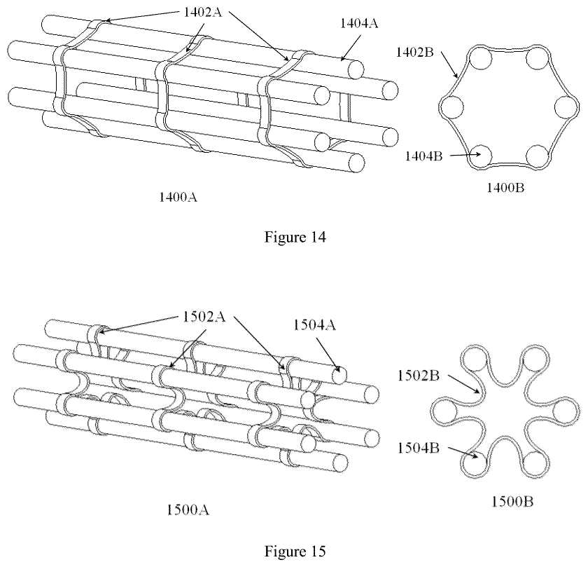

In another embodiment, FIG. 14, illustrates the coaxial dilation speculum apparatus in a perspective view 1400A and an axial view 1400B with three quasi-circular-springs 1402A-1402B coupled to internally captured curvilinear beams 1404A-1404B. The coaxial dilation speculum apparatus 1400A-1400B is illustrated in the expanded configuration. The coaxial dilation speculum apparatus 1400A-1400B being a conformable cylindrical cage constructed of three quasi-cylindrical springs 1402A-1402B that have features in-built into their periphery to appropriately capture the curvilinear beams 1404A-1404B so as to evenly distribute them on the circumference. This assembly 1400A-1400B in effect dilates and supports, radially and longitudinally, the surrounding vaginal walls 102 while enabling an unobstructed view and path for operator-physician managed instruments to probe and operate on the vaginal canal 102 and the cervix 106.

The vaginal canal 102, is a muscular quasi-cylindrical tissue which is highly elastic and in a contracted state under normal conditions. To introduce the speculum into the introitus 108 the speculum 1400A-1400B needs to be also apriori contracted-retracted. Referring to FIG. 15, it illustrates the coaxial dilation speculum apparatus 1400A-1400B in a contracted-retracted state in a perspective view 1500A and an axial view 1500B with three quasi-cylindrical springs 1502A-1502B coupled to six externally captured curvilinear beams 1504A-1504B. Over and above supporting the walls of the vaginal canal 102, the curvilinear beams 1504A-1504B also serve the additional purpose of facilitating capture and guidance of instruments to be described later in the description.

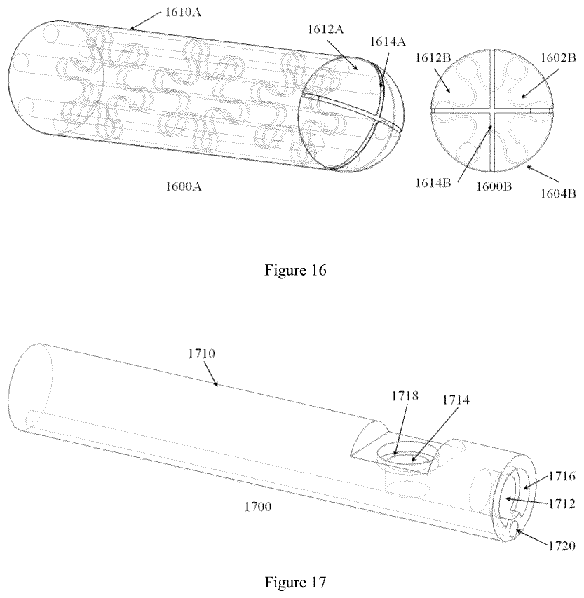

The coaxial dilation speculum apparatus 1500A-1500B is held in the contracted-retracted state by inserting it into a cylindrical sheath-cover with a hemispherical scribed tip 800 as illustrated in FIG. 8, in a perspective view 800A and an axial view 800B. FIG. 16 illustrates an assembly of the coaxial dilation speculum apparatus 1500A-1500B inserted into the sheath-cover 800A-800B in a perspective view 1600A and an axial view 1600B that comprises three quasi-cylindrical springs 1602B coupled to six internally captured curvilinear beams 1604B in a contracted-retracted state inserted in a cylindrical sheath-cover 1610A with a hemispherical tip 1612A-1612B that is locally scribed 1614A-1614B. After the operator locates, adjusts, and places the speculum apparatus assembly 1600A-1600B into the vaginal canal 102, the cylindrical sheath cover with a hemispherical tip 1610A-1612A is removed. Upon removal of the sheath cover, the coaxial dilation speculum expands due to the release of the compressed spring force and dilates the vaginal canal 102 in conformity with its anatomy, thus enabling an unobstructed view and path for operator-physician managed instruments to probe and operate on the vaginal canal 102 and the cervix 106. Here the quasi-cylindrical springs 1602B and curvilinear beams 1604B geometries are selected to spread the dilation load so as to reduce pressure concentration on the vaginal canal 102 tissues. Circular curvilinear beam 1604B geometries, six in number are presented to illustrate feasibility. It is understood that other beam geometries and numbers may also be utilized without departing from the spirit or scope of the present disclosure. Similarly, the three quasi-cylindrical springs 1602B with six lobe geometry is presented to illustrate feasibility. It is understood that other quasi-cylindrical spring geometries and lobe numbers may also be utilized without departing from the spirit or scope of the present disclosure. The description is, therefore, not to be taken in a limiting sense.

Referring to FIG. 17, it illustrates a perspective view 1700 of the assembly of the camera module 1710. The visualization of the vaginal canal 102 is facilitated by dual high resolution digital cameras with dedicated adjustable intensity light sources. The first camera 1712 provides a view directly in front and axially. The second camera 1714 provides a view radially or sideways, i.e., to the lateral walls of the vaginal canal 102. The camera provides snapshot and video access-visibility that is characterized by black-white, color, low-light, or IR visibility, high resolution capability, depth of field adjustment, extended field of view adjustment, autofocus capability, and zoom capability. The camera module 1710 then remotes the image via cellular, Wi-Fi, BT, or wired USB via a wired connection to a user selectable display device including mobile devices, computers, smart TVs, and-or AR-VR glasses. Further included are lighting features providing sufficient illumination to allow for the ease of view with the camera of the extended region internal to the vaginal canal 102. The lighting feature comprises light sources being either or combination of LEDs, laser, incandescent, or fiber optic remotely piped in, where the light intensity is adjustable. The light source(s) illuminate in the axial 1716 and sideways 1718 directions. The system of optics, lenses, optical fibers being in-situ within or integrated within the camera module 1710 is shown. Further, the camera module has features 1720 to introduce a jet of gas to provide a puff of gas or a jet-spray of liquid to cleanse-push away or irrigate any refuse-secretions present on the vaginal canal 102 and cervix 106 surfaces.

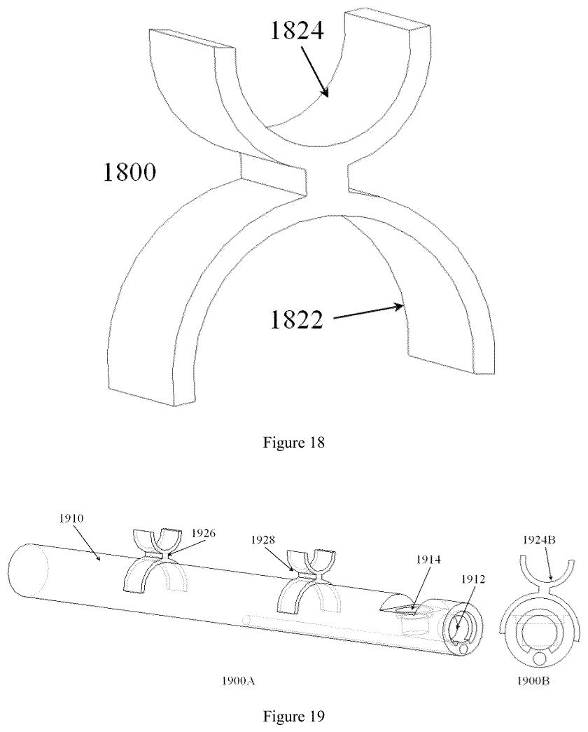

FIG. 18 illustrates a perspective view 1800 of a supporting clip. One clasp 1822 of the clip attaches and holds on to the camera module and the second clasp 1824 of the clip attaches to any of the curvilinear beams in the coaxial dilation speculum apparatus.

FIG. 19 illustrates a perspective view 1900A and an axial view 1900B of the assembly of the camera module 1910 with two support clips 1926 and 1928. The visualization of the vaginal canal 102 is facilitated by dual high resolution digital cameras with dedicated adjustable intensity light sources. The first camera 1912 provides view directly in front and axially. The second camera 1914 provides a view radially or sideways, i.e., to the lateral walls of the vaginal canal 102. These clips allow for rotational and longitudinal traversal of the camera according to the desired position by the operator-physician.

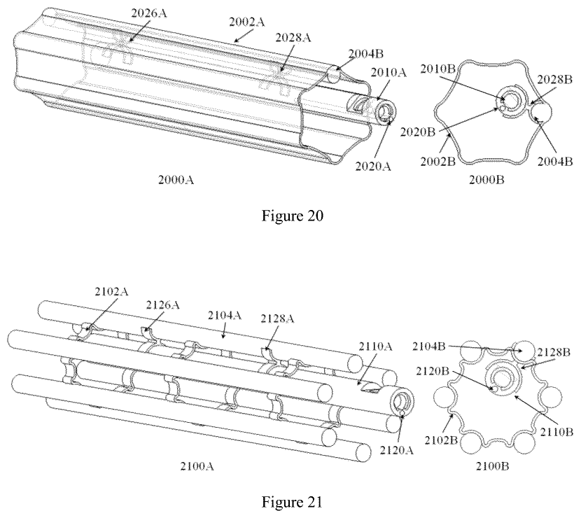

FIG. 20 illustrates a perspective view 2000A and an axial view 2000B of a camera module assembled with the expanded coaxial dilation speculum apparatus with a quasi-cylindrical corrugated monocoque spring 2002A-2002B to which is attached a curvilinear beam 2004A-2004B. FIG. 20 further illustrates a method of introducing the camera module 2010A-2010B into the speculum apparatus and traversing the entire vaginal canal facilitated by the following features in the design. The cervix 106 at the distal end of the vaginal canal 102 is unobstructed by the open distal end of the speculum apparatus in the expanded state. The camera module is attached to two support clips 2026 and 2028. These clips allow for rotational and longitudinal traversal of the camera module. The two clips 2026, 2028 are further clipped onto the curvilinear beam 2004A-2004B again allowing for rotational and longitudinal traverse of the camera module. Not only is the camera rotatable with traverse in-out, but so also is the coaxial dilation speculum apparatus. Further, there is a lot of space to introduce instrumentation for any diagnostic and surgical procedures. Additionally, for cleaner visualization the camera module has features 2020A-2020B to introduce a jet of gas to provide a puff of gas or a jet-spray of liquid to cleanse-push away or irrigate any refuse-secretions present on the vaginal canal 102 and cervix 106 surfaces.

FIG. 21 illustrates a perspective view 2100A and an axial view 2100B of a camera module assembled with the expanded coaxial dilation speculum apparatus with three quasi-cylindrical springs 102A-2102B coupled to externally captured curvilinear beams 2104A-2104B. The coaxial dilation speculum apparatus 2100A-2100B is illustrated in the expanded configuration. FIG. 21 further illustrates a method of introducing the camera module 2110A-2110B into the speculum apparatus and traversing the entire vaginal canal facilitated by the following features in the design. The cervix 106 at the distal end of the vaginal canal 102 is unobstructed by the open distal end of the speculum apparatus in the expanded state. The camera module is attached to two support clips 2126A and 2128-A-2128B. These clips allow for rotational and longitudinal traversal of the camera module. The clips 2126A-2126B are further clipped onto one of the curvilinear beams 2104A-2104B again allowing for rotational and longitudinal traverse of the camera module. Not only is the camera rotatable with traverse in-out, but so also is the coaxial dilation speculum apparatus. Further, there is a lot of space to introduce instrumentation for any diagnostic and surgical procedures. Additionally, for cleaner visualization the camera module has features 2120A-2120B to introduce a jet of gas to provide a puff of gas or a jet-spray of liquid to cleanse-push away or irrigate any refuse-secretions present on the vaginal canal 102 and cervix 106 surfaces.

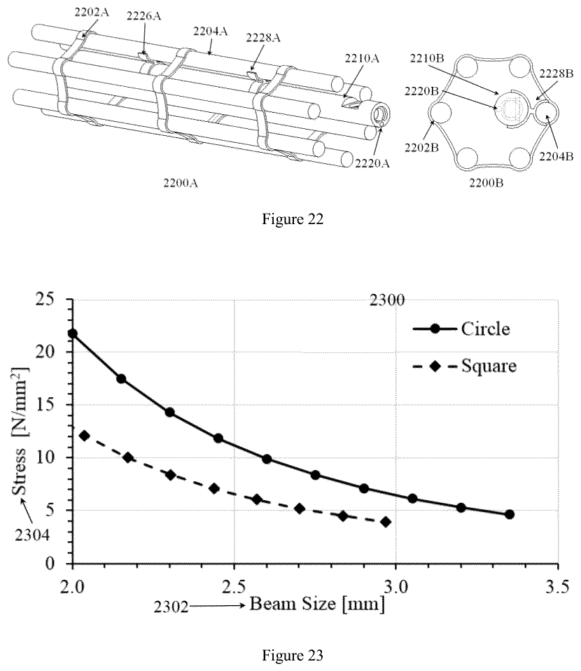

FIG. 22 illustrates a perspective view 2200A and an axial view 2200B of a camera module assembled with the expanded coaxial dilation speculum apparatus with three quasi-cylindrical springs 2202A-2202B coupled to internally captured curvilinear beams 2204A-2204B. The coaxial dilation speculum apparatus 2200A-2200B is illustrated in the expanded configuration. FIG. 22 further illustrates a method of introducing the camera module 2210A-2210B into the speculum apparatus and traversing the entire vaginal canal facilitated by the following features in the design. The cervix 106 at the distal end of the vaginal canal 102 is unobstructed by the open distal end of the speculum apparatus in the expanded state. The camera module is attached to two support clips 2226A and 2228-A-2228B. These clips allow for rotational and longitudinal traversal of the camera module. The clips 2226A-2228A are further clipped onto one of the curvilinear beams 2204A-2204B again allowing for rotational and longitudinal traverse of the camera module. Not only is the camera module rotatable with traverse in-out, but so also is the coaxial dilation speculum apparatus. Further, there is a lot of space to introduce instrumentation for any diagnostic and surgical procedures. Additionally, for cleaner visualization the camera module has features 2220A-2220B to introduce a jet of gas to provide a puff of gas or a jet-spray of liquid to cleanse-push away or irrigate any refuse-secretions present on the vaginal canal 102 and cervix 106 surfaces.

### Human Vagina Size and Force Statistics

Table 1. provides the physical size range statistics of the human female pelvic region specifically the introitus 108 diameter T108 that is from 24 mm to 65 mm (0.94″ to 2.6″), and the vaginal canal 102 length T102 that is from 68 mm to 148 mm (2.7″ to 5.8″). The muscle force produced by the vaginal canal 102 muscles in healthy human females is of the order of 4 pound-force or 18.2 Newtons. Hence, the coaxial dilation speculum apparatus is designed with the following limits, a contracted-retracted diameter of about 18 mm, a maximum expanded diameter of about 65 mm, a total circumferential force of about 5 pound-force or 22 Newtons, and a length of about 200 mm. The length is designed long enough with sufficient of the coaxial dilation speculum apparatus external to the vaginal introitus 108 opening so as permit the operator-physician to physically grasp and orient the coaxial dilation speculum apparatus circumferentially, orient the coaxial dilation speculum apparatus longitudinally, attach the camera and light source to the internal curvilinear beam of the expanded coaxial dilation speculum apparatus, and to contract-retract and extract the coaxial dilation speculum apparatus. The total force needed to contract-retract the coaxial dilation speculum apparatus is about 5 pound-force or 22 Newtons, which is simple to perform in a single hand-held squeeze operation. Studies indicate that between men and women, their grip strength is in the range of 185 Newtons-460 Newtons which is more than tenfold required for this task.

FIG. 23 illustrates a relation 2300 between the physical geometrical size 2302 and shape (circle, square) of the curvilinear beam(s) 504A-504B and the stress 2304 induced in its material makeup. The quasi-cylindrical springs 502A-502B and curvilinear beam(s) 504A-504B take the load of pushing against and dilating the lateral walls of the vaginal canal 102. The total maximum load is expected to be about 5 pound-force or 22 Newtons. This load is distributed across the set of quasi-cylindrical springs 502A-502B and curvilinear beam(s) 504A-504B. Hence, for example for a speculum with six curvilinear beams, the load per curvilinear beam is approximately 4 Newtons each. If we use three quasi-cylindrical springs to support the curvilinear beams, assuming a 150 mm-6″ long coaxial dilation speculum apparatus, the maximum span between each curvilinear beam is 50 mm-2″. Depending on the physical geometrical size 2302 and shape (circle, square) of the curvilinear beam, the stress 2304 induced in the curvilinear beam material is presented in FIG. 23.

Table 2 enumerates the physical properties T200 for materials of which the curvilinear beam(s) 504A-504B and quasi-cylindrical springs 502A-502B may be made. The materials T202, plastics T206 and metals T208, indicate their ability to support high levels of stress T204, specifically that metals T208 are a lot stiffer, able to withstand stresses greater than 10 times T212 that plastics can support T210. In other words, the use of metals T208 enable the support of a lot more force for a lot smaller deflections or alternatively, thinner curvilinear beam(s) 504A-504B structures. Several materials exist that support the envisaged designs of the quasi-cylindrical springs 502A-502B and the curvilinear beam(s) 504A-504B. Given the curvilinear beam geometrical size 2302 and shape (circle, square) range and the material physical properties T200, it is evident that the use of both plastics and metals can support the stresses 2304, thus validating the feasibility of the coaxial dilation speculum apparatus design.

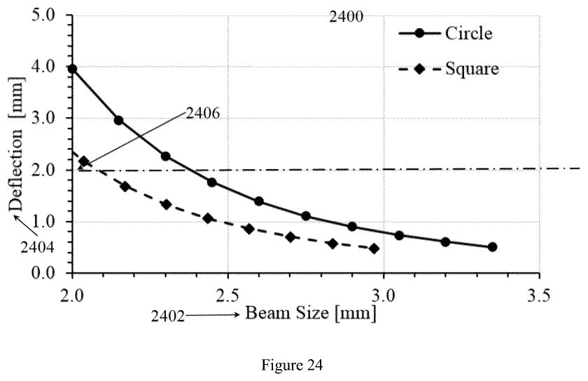

FIG. 24 illustrates a relation 2400 between the geometrical size 2402 and shape (circle, square) of the curvilinear beam(s) 504A-504B and the sag-bend 2404 induced. The sag-bend 2404 then of the curvilinear beam, if plastic is used, is provided in FIG. 24 for circular and square cross-sections. Given that we would like the sag-bend 2404 to be less than a few millimeters 2406, it would mean that we use a curvilinear beam of approximately 2 mm in diameter 2402 or more.

The above analysis validates the structural feasibility of the coaxial dilation speculum apparatus to dilate the vaginal wall while providing ample physical and visual access to the vaginal canal 102. It is intended that the geometries used will impose minimum impact on the tissues of the vaginal canal 102, partly by proper surface treatments, such as polished and lubricated. Further, geometries are selected to spread the distention load so as to reduce pressure concentration on the tissues.

In the above description of exemplary embodiments of the curvilinear beams, of circular or rectangular geometries, are presented to illustrate feasibility. It is understood that other beam geometries may also be utilized without departing from the spirit or scope of the present disclosure. The description is, therefore, not to be taken in a limiting sense.

In the above description of exemplary embodiments of the quasi-cylindrical springs, flat-ribbon geometries are presented to illustrate feasibility. It is understood that other spring geometries may also be utilized without departing from the spirit or scope of the present disclosure. The description is, therefore, not to be taken in a limiting sense.

Those of ordinary skill in the art will appreciate that the hardware components may vary and are not intended to be exhaustive, but rather are representative to highlight essential components that are utilized to implement aspects of the described embodiments. For example, other devices-components may be used in addition to or in place of the hardware and-or firmware depicted. The example is not meant to imply architectural or other limitations with respect to the presently described embodiments and-or the general invention.

In the above description of exemplary embodiments of the disclosure, specific exemplary embodiments in which the various aspects of the disclosure may be practiced are described in sufficient detail to enable those skilled in the art to practice the invention, and it is to be understood that other embodiments may be utilized and that logical, architectural, programmatic, mechanical, electrical and other changes may be made without departing from the spirit or scope of the present disclosure. The description is, therefore, not to be taken in a limiting sense.

It is understood that the use of specific component and-or device names, such as those described herein, are for example only and not meant to imply any limitations on the described embodiments. Thus, each term utilized herein is to be given its broadest interpretation given the context in which that term is utilized.

### Method of the Invention

The vaginal canal 102 in its normal unexcited state is almost a retracted-collapsed wall of muscle tissue. In reality, it is an expandable quasi-cylindrical tubular shell, which with appropriate and careful physical intervention, can be expanded. Hence, there is an opportunity to insert a speculum into the vaginal canal 102 in a retracted-contracted state and after introduction cause it to expand so as to dilate the tissue of the vaginal canal walls.

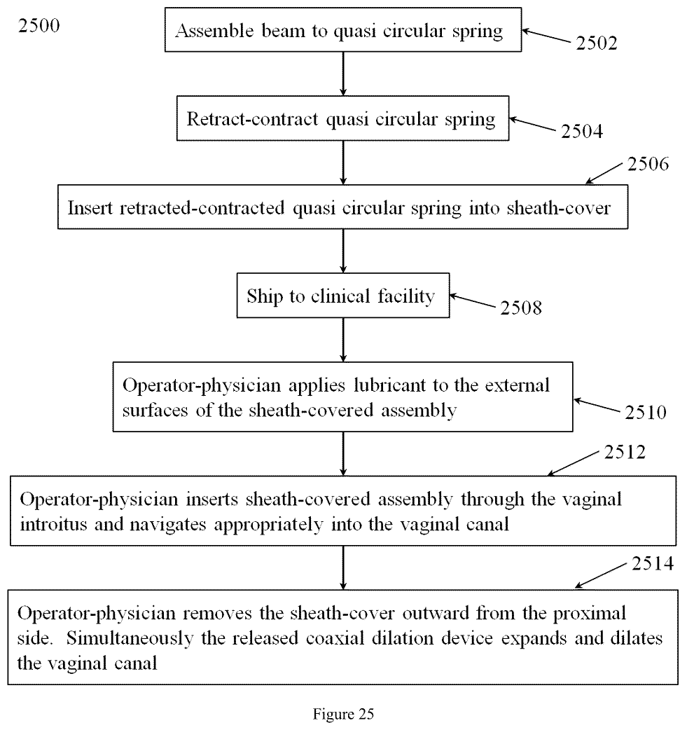

Referring to FIG. 25, the following method 2500 is proposed to prepare the coaxial dilation speculum apparatus. It is envisaged that at the manufacturing facility, the coaxial quasi-circular-spring(s) 502A are assembled 2502 with the requisite number of curvilinear beam(s) 504A. The assembly 500A is then arranged 2504 in a contracted-retracted arrangement 500B, at an appropriate diameter that is acceptable for ease of insertion. The contracted-retracted arrangement 500B is then inserted 2506 into a sheath-cover 800A. The resulting assembly 1000A-1300A-1600A is shipped 2508 to the clinics for operator-physician use. In the event of a vaginal examination-procedure, the operator-physician would prior to insertion of the contracted-retracted arrangement 1000A-1300A-1600A, lubricates 2510 the external surface of the cover-sheath 1010A appropriately to facilitate ease of insertion. Upon insertion 2512 through the introitus 108 and navigating to an adequate position, the sheath-cover 1010A will be removed 2514 by the operator-physician by pulling the sheath-cover 810A outward in the proximal direction. Following removal of the cover-sheath 910, the contracted-retracted dilation assembly 700A-1200A-1500A finds itself no more restrained by the sheath-cover 810A and hence expands to conform to the anatomy (length, diameter, and shape) of the vaginal canal 102.

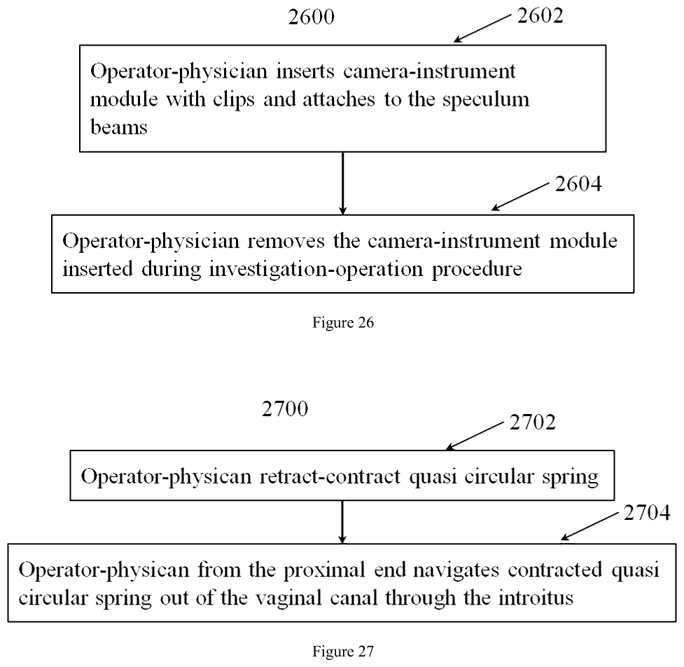

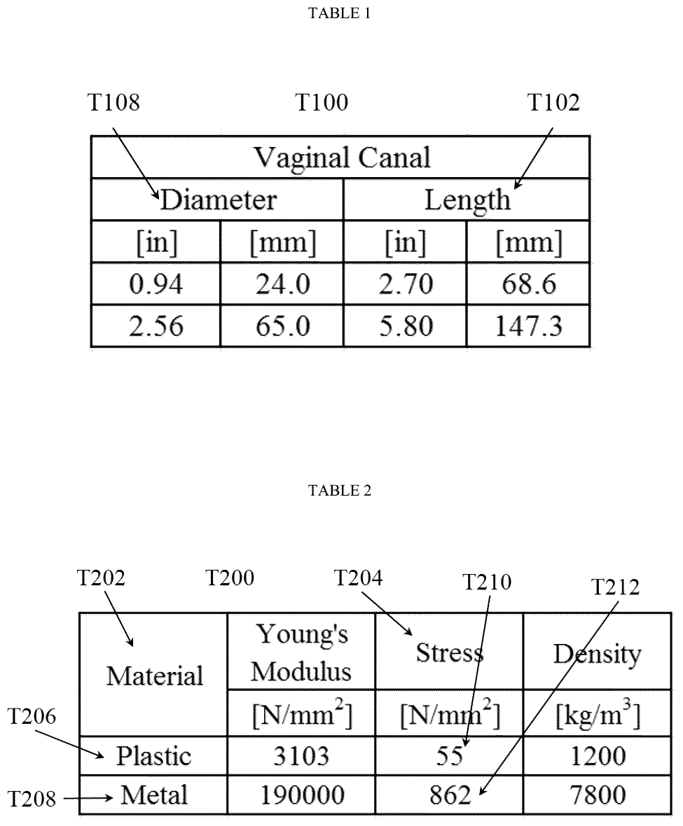

Referring to FIG. 26, the following method 2600 is proposed to insert instrument modules 2602 like the camera module 1710. The operator-physician installs two or more clips 1800 on the camera module 1700. This assembly 1900A-1900B is then inserted into the expanded coaxial quasi-cylindrical spring device 600A-1100A-1400A and clipped onto the appropriate curvilinear beam within to arrive at 2000A-2100A-2200A arrangement. On the completion of the medical procedures, the operator-physician would remove instrument modules 2604 by unclipping the camera and clip assembly 1900A-1900B from the curvilinear beam within the expanded coaxial quasi-cylindrical spring apparatus 600A-1100A-1400A and then removing the module from the proximal end of the expanded coaxial quasi-cylindrical spring apparatus 600A-1100A-1400A.

Referring to FIG. 27, the following method 2700 is proposed to remove the expanded coaxial quasi-cylindrical spring apparatus 600A-1100A-1400A from the vaginal canal 102. On the completion of the medical procedures, the operator-physician would contract-retract 2702 the quasi-cylindrical spring apparatus 600A-1100A-1400A. Then the operator-physician would navigate 2704 the contracted-retracted quasi-cylindrical spring apparatus 700A-1200A-1500A in the outward proximal direction and exit it out of the introitus 108.

What is claimed is:

1. A vaginal speculum, comprising:
a sheath-cover adapted for gripping by an operator-physician; and
a coaxial dilation speculum apparatus, for expanding the entire vaginal canal and visualization of the distal end and lateral walls of the vaginal canal, having longitudinal curvilinear beams and quasi-cylindrical springs, wherein the longitudinal curvilinear beams are supported by the quasi-cylindrical springs that when inserted into the vaginal canal and following removal of the sheath-cover, they expand, the coaxial dilation speculum apparatus having a proximal end and a distal end, wherein the distal end facilitates smooth insertion and navigation within the vaginal canal to adjust the depth of insertion, and the proximal end facilitates holding, insertion, and rotation of the coaxial dilation speculum apparatus, and the coaxial dilation speculum apparatus on expanding facilitates adequate visualization and expansion of the vaginal canal.

2. A speculum as set forth in claim 1, wherein the coaxial dilation speculum apparatus comprises the quasi-cylindrical monocoque spring which is the entire length of the coaxial dilation speculum apparatus, and the curvilinear beam that is attached internal to a quasi-cylindrical monocoque spring to allow upon insertion and removal of the sheath-cover uniform expansion of the coaxial dilation speculum apparatus in conformity to the anatomy of the vaginal canal.

3. A speculum as set forth in claim 1, wherein the coaxial dilation speculum apparatus comprises at least two quasi-cylindrical springs, and at least three curvilinear beams that are attached external to the quasi-cylindrical springs, the quasi-cylindrical springs are uniformly spaced lengthwise along the curvilinear beams, and the curvilinear beams are circumferentially spaced on the quasi-cylindrical springs to allow upon insertion and removal of the sheath-cover an uniform expansion of the coaxial dilation speculum apparatus in conformity to the anatomy of the vaginal canal.

4. A speculum as set forth in claim 1, wherein the coaxial dilation speculum apparatus comprises at least two quasi-cylindrical springs, and at least three curvilinear beams that are attached internal to the quasi-cylindrical springs, the quasi-cylindrical springs are uniformly spaced lengthwise along the curvilinear beams and the curvilinear beams are circumferentially spaced on the quasi-cylindrical springs to allow upon insertion and removal of the cover sheath uniform expansion of the coaxial dilation speculum apparatus in conformity to the anatomy of the vaginal canal.

5. A speculum as set forth in claim 1, wherein the quasi-cylindrical springs are made of a spring material that facilitates expansion of the coaxial dilation speculum apparatus upon insertion into the entire vaginal canal after removal of the sheath-cover.

6. A speculum as set forth in claim 1, wherein the quasi-cylindrical springs and the curvilinear beams are made of a plastic material.

7. A speculum as set forth in claim 1, wherein the quasi-cylindrical springs and the curvilinear beams are made of a metal material.

8. A speculum as set forth in claim 1, wherein the sheath-cover is made of a plastic material.

9. A speculum as set forth in claim 1, wherein the sheath-cover comprises a cylindrical tube having a proximal end that is open, and a hemispherical tip at the distal end, the hemispherical tip having pre-formed scribes, wherein the scribes preferentially rupture on removal towards the proximal end of the cover sheath by the operator-physician.

10. A speculum as set forth in claim 1, further comprising an imaging apparatus, the imaging apparatus includes at least one camera and at least one light source, wherein the imaging apparatus is attached to the curvilinear beams having supporting clips, the camera(s) facilitates high resolution image, and the light source facilitates adjustable light intensity.

11. A speculum as set forth in claim 1, further comprising a conduit to introduce a jet of gas or introduce a jet of liquid to cleanse or irrigate any refuse-secretions present on the vaginal canal and cervix surfaces.

12. A speculum as set forth in claim 1, wherein the coaxial dilation speculum apparatus is centered about an axis extending from the distal end to the proximal end, and accommodating to the length, diameter, and shape of the vaginal canal, the proximal end is protruding externally through the vaginal introitus to allow the operator-physician to locate the depth and direction of the dilation assembly in the vaginal canal.

13. A method for use in performing a medical procedure, comprising:
a sheath-cover and a coaxial dilation speculum apparatus, the coaxial dilation speculum apparatus including a default, contracted-retracted position and an expanded position;
introducing a distal end of the coaxial dilation speculum apparatus into the vaginal introitus with the coaxial dilation speculum apparatus in the default contracted-retracted position;
navigating the coaxial dilation speculum apparatus in the vaginal canal to an appropriate position;
removing the sheath cover from the coaxial dilation speculum apparatus and transforming the coaxial dilation speculum apparatus from the default, contracted-retracted position to the expanded position;
inserting and attaching an imaging apparatus for visualization of the vaginal canal;
removing the imaging apparatus from the dilation assembly upon completion of the procedure;
removing the coaxial dilation speculum apparatus upon completion of the procedure.

14. The method as in claim 13, wherein the introducing step includes providing the sheath-cover in a generally tubular shape with a distal end that is closed and a proximal end that is open, the distal end shaped to facilitate smooth insertion and navigation within the vaginal canal.

15. The method as in claim 13, wherein the introducing step includes providing the coaxial dilation speculum apparatus comprising a material with sufficient strength to maintain the vaginal canal in the expanded state.

16. The method as in claim 13, wherein the introducing step includes providing the sheath-cover comprising a material with sufficient strength to maintain the coaxial dilation speculum apparatus in the default contracted state.

17. The method in claim 13, further comprising the sheath cover encompassing the coaxial dilation speculum apparatus in the contracted position in the introducing step and removed in the removal step while the coaxial dilation speculum apparatus remains in the vaginal canal in an expanded state.

18. The method as in claim 13, further comprising providing a lubricant on the sheath-cover before the introducing step.

19. The method as in claim 13, wherein the transforming step includes pulling the sheath-cover in an axial direction away from the distal end towards the proximal end such that the closed distal end ruptures to allow the sheath cover to be pulled away and removed from the coaxial dilation speculum apparatus.

20. The method as in claim 13, wherein the transforming step includes pulling the sheath-cover in an axially direction away from the vaginal canal while the coaxial dilation speculum apparatus is in the default contracted position and the closed distal end ruptures to allow the sheath-cover to be removed while the coaxial dilation speculum apparatus moves to the expanded position.
